(19) United States
(12) Patent Application Publication
(10) Pub. No.: US 2021/0121660 A1
(43) Pub. Date: Apr. 29, 2021

(54) EXPANDABLE VASCULAR INTRODUCER

(71) Applicant: Oscor Inc., Palm Harbor, FL (US)

(72) Inventors: Thomas P. Osypka, Palm Harbor, FL (US); Andrew J. Enerson, New Port Richey, FL (US); Jake Gerber, Clearwater, FL (US)

(73) Assignee: Oscor Inc., Palm Harbor, FL (US)

(21) Appl. No.: 17/077,087

(22) Filed: Oct. 22, 2020

Related U.S. Application Data
(60) Provisional application No. 62/926,838, filed on Oct. 28, 2019.

(51) Int. Cl. A61M 25/00

(52) U.S. Cl. CPC A61M 25/0023; A61M 2025/0024

(57) ABSTRACT

A vascular introducer is disclosed that includes a proximal housing defining an axial passageway, a body portion extending distally from the proximal housing and defining an axial bore in communication with the axial passageway of the proximal housing, and an elongated tubular sheath extending distally from the axial bore of the body portion and defining a central lumen, wherein the axial bore of the body portion and the central lumen of the tubular sheath are adapted and configured to transition between a first inner diameter and a second inner diameter in response to introduction of a dilator having an outer diameter that is greater than the first inner diameter of the axial bore of the body portion and the central lumen of the tubular sheath.

### CROSS-REFERENCE TO RELATED APPLICATIONS

This application claims the benefit of priority to U.S. Provisional Patent Application Ser. No. 62/926,838, filed Oct. 28, 2019, the disclosure of which is incorporated herein by reference in its entirety.

### BACKGROUND OF THE INVENTION

### 1. Field of the Invention

The subject invention is directed to surgical instrumentation, and more particularly, to a vascular introducer that includes a dynamically expandable sheath which expands and contracts in response to the introduction of different sized dilators.

### 2. Description of Related Art

Vascular access devices for minimally invasive percutaneous interventional procedures, including endovascular procedures are known in the art. To reduce trauma, access devices having a relatively small sheath size are typically employed during a percutaneous procedure.

However, some percutaneous interventional procedures involve the use of large devices that require relatively large sheaths to deliver them to the intended site in the body. Sheath sizes for such procedures can vary from 14F to 24 F (0.184″ to 0.315″). As a result, access site trauma can increase, which may lead to vessel damage, bleeding and increased risk of infection.

To address these issues, access device have been developed with sheaths that can be expanded within the body so that their removal from the operative site is not required to upsize, as disclosed for example in U.S. Pat. No. 9,301,840 to Nguyen et al. or U.S. Pat. No. 9,555,214 to Ren et al. Still, there is a need for more advanced and improved expandable sheaths. The subject invention satisfies that need.

### SUMMARY OF THE DISCLOSURE

The subject invention is directed to a new and useful vascular introducer that includes a proximal housing defining an axial passageway, a body portion extending distally from the proximal housing and defining an axial bore in communication with the axial passageway of the proximal housing, and an elongated tubular sheath extending distally from the axial bore of the body portion and defining a central lumen.

The axial bore of the body portion and the central lumen of the tubular sheath are adapted and configured to transition or otherwise expand between a first inner diameter and a second inner diameter in response to the introduction of a dilator having an outer diameter that is greater than the first inner diameter of the axial bore of the body portion and the central lumen of the tubular sheath.

Preferably, the proximal housing portion includes a proximal flange configured for engagement within a proximal end of the axial bore of the body portion for easy assembly. The body portion is formed from a flexible material and the axial bore of the body portion tapers radially inwardly in a distal direction to aid in diametrical expansion.

The tubular sheath includes an outer sheath portion and an inner sheath portion. The outer sheath portion is defined by an elongated flexible tube and the inner sheath portion is defined by a scored extrusion. The scored extrusion is preferably formed from HDPE or a similar material such as a Polyamide or other similar Nylon material. The scored extrusion that defines the inner sheath portion of the tubular sheath includes a plurality of circumferentially spaced apart elongated struts that are separated from one another by scored longitudinal grooves, and each of the struts has an arcuate inner surface.

In a preferred embodiment of the invention, the scored extrusion includes twelve (12) circumferentially spaced apart struts. It is envisioned however, that there can be fewer than 12 struts or more than 12 struts in the scored extrusion. For example, the scored extrusion can have four (4) spaced apart struts, which can provide well balanced expansion of the outer sheath portion.

Preferably, the axial bore of the body portion and the central lumen of the tubular sheath is adapted and configured to transition between a first inner diameter of 14F and a second inner diameter of 18F. Those skilled in the art will readily appreciate the first inner diameter of the tubular sheath can be greater or less than 14F, and that the second inner diameter of the tubular sheath can be greater or less than 18F, without departing from the spirit or scope of the subject disclosure.

The proximal housing preferably has an end cap that supports a hemostatic seal for sealing the axial passageway. It is envisioned that the housing and end cap can be splittable into two halves along a diametrical parting line to aide in removing the sheath from the operative site. In such an instance, the tubular sheath would also be splittable along sore lines formed in the outer sheath portion.

It is also envisioned that the vascular introducer system of the subject invention may be provided for use and/or sale as a kit that would include an enclosure or sealed tray containing a dynamically expandable vascular introducer as described above, and at least one dilator having an outer diameter ranging from about 14F to about 18F.

These and other features of the vascular introducer system of the subject invention will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the vascular introducer system of the subject invention without undue experimentation, preferred embodiments thereof will be described in detail herein below with reference to the figures wherein:

FIG. 1 is a perspective view of a vascular introducer constructed in accordance with a preferred embodiment of the subject invention, wherein a portion of the tubular sheath is shown in cross-section to reveal the central lumen thereof;

FIG. 2 is a side elevational view of the vascular introducer of FIG. 1, with a dilator partially inserted therein, and wherein a portion of the tubular sheath is shown in cross-section to illustrate how the tubular sheath is dynamically expanded by the shaft of the dilator from a first inner diameter to a second inner diameter;

FIG. 3 is an enlarged localized view taken from FIG. 2;

FIG. 4 is a side elevational view of the proximal housing of the vascular introducer of FIG. 1;

FIG. 5 is a perspective view of the proximal housing of the vascular introducer of FIG. 1;

FIG. 6 is a side elevational view of the body portion of the vascular introducer of FIG. 1;

FIG. 7 is a perspective view of the body portion of the vascular introducer of FIG. 1;

FIG. 8 is an enlarged localized view of taken from FIG. 2, illustrating the connection between the proximal flange of proximal housing and the axial bore of the body portion;

FIG. 9 is a cross-sectional view of the tubular sheath showing, among other dimensions, the first inner diameter of the central bore; and

FIG. 10 is a cross-sectional view of the fully expanded tubular sheath showing, among other dimensions, the second inner diameter of the central bore.

### DETAILED DESCRIPTION OF THE PREFERRED EMBODIMENTS

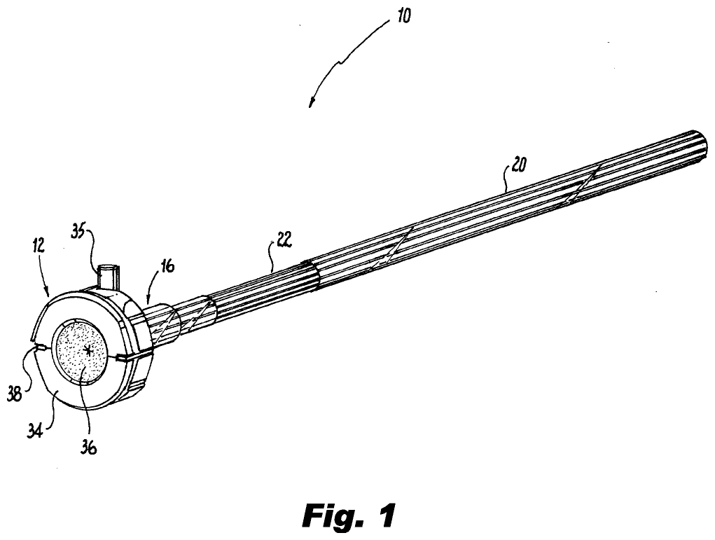

Referring now to the drawings wherein like reference numeral identify similar structural elements or features of the subject invention, there is illustrated in FIG. 1 a new and useful vascular introducer constructed in accordance with a preferred embodiment of the subject invention and designated generally by reference numeral 10. In brief, the vascular introducer 10 is designed to be expanded within the body so that its removal from the operative site is not required to accommodate the placement of a larger sheath to introduce a larger sized instrument or device during a percutaneous interventional procedure.

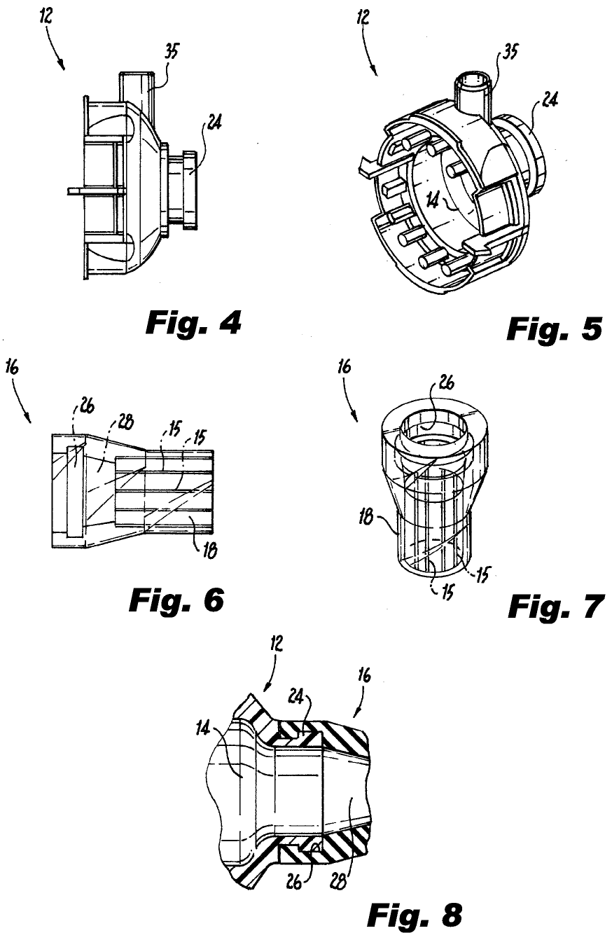

The vascular introducer 10 includes a proximal housing 12 defining an axial passageway 14 (see FIGS. 5 and 8), a body portion 16 extending distally from the proximal housing 12 and defining an axial bore 18 (see FIGS. 6 and 7) in communication with the axial passageway of the proximal housing, as best seen in FIG. 8. The vascular introducer 10 further includes an elongated tubular sheath 20 that extends distally from the axial bore 18 of the body portion 16 and defines a central lumen 22, as best seen in FIG. 2.

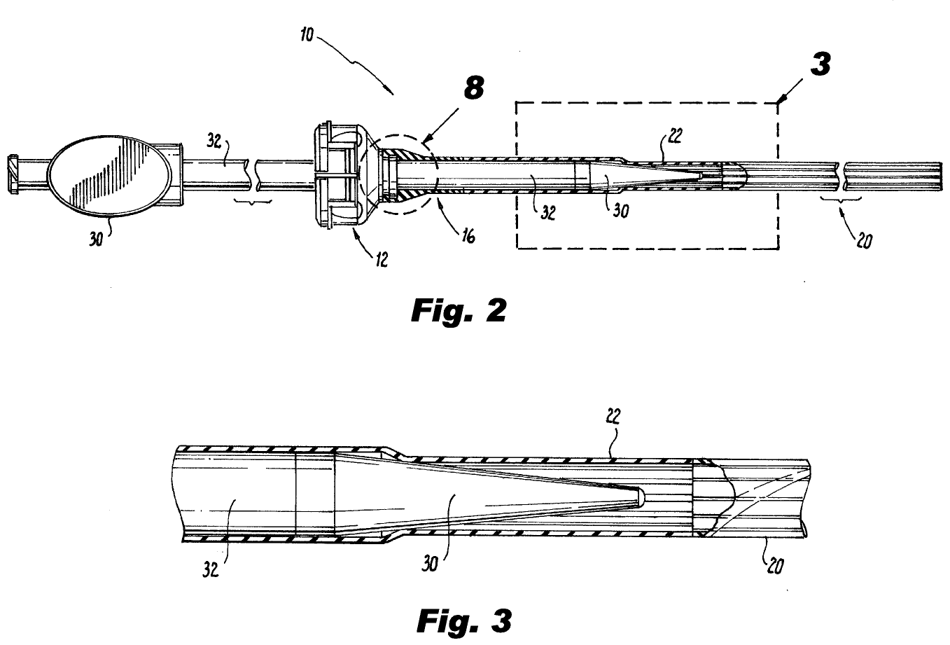

As explained in more detail below and as illustrated in FIGS. 2 and 3, the axial bore 18 of the body portion 16 and the central lumen 22 of the tubular sheath 20 are adapted and configured to transition or otherwise expand between a first inner diameter ID₁ and a second inner diameter ID₂ in response to the introduction of a dilator 30 having a shaft 32 with an outer diameter OD that is greater than the first inner diameter ID₁ of the axial bore 18 of the body portion 16 and the central lumen 22 of the tubular sheath 20. The dilator 30 can include a central bore for accommodating a guidewire or a stylet, and the dilator shaft 32 has a tapered distal tip for facilitating its percutaneous insertion.

Referring to FIGS. 4 through 8, the proximal housing portion 12 of vascular introducer 10 includes a proximal flange 24 configured for engagement within a radially enlarged recessed section 26 of the axial bore 18 of the body portion 16 for easy assembly.

The body portion 16 is formed from a flexible material, such as silicone or another similar bio-compatible elastomeric material, and the axial bore 18 of the body portion 16 has a radially inwardly tapered section 28 that aids in diametrical expansion of the axial bore 18, as best seen in FIG. 8. In addition, the body portion 16 has internal strain relief features 15 best seen in FIGS. 6 and 7, to accommodate the radial expansion of the axial bore 18.

As best seen in FIG. 1, the proximal housing 12 of vascular introducer 10 preferably has an end cap 34 that supports a hemostatic seal 36 for fluidly sealing the axial passageway 14 that communicates with the central lumen 22 of the tubular sheath 20. In addition, the proximal housing 12 of vascular introducer 10 includes a radially extending hub 35 for accommodating a 3-way stopcock valve, which is not shown.

It is envisioned that the proximal housing 12 and the end cap 34 can be splittable into two halves along a diametrical parting line 38 to aide in removing the tubular sheath 20 from the operative site without having to remove an instrument or device extending therethrough. In such an instance, the tubular sheath 20 would also be splittable along its length.

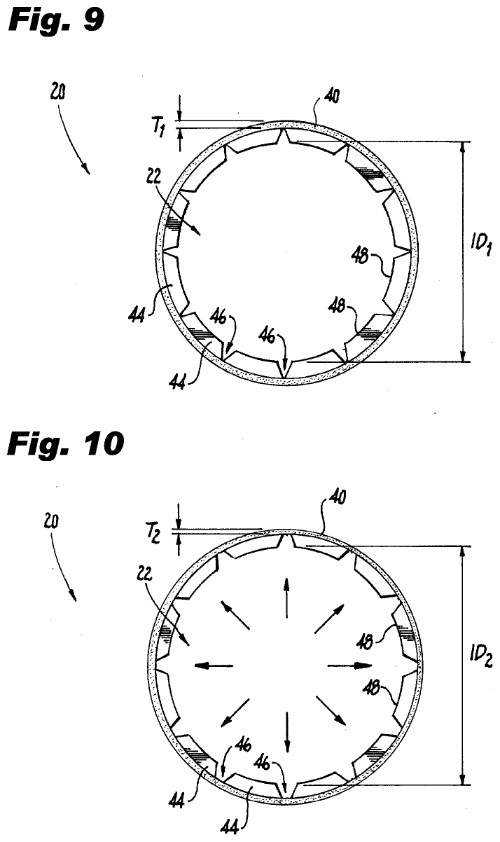

Referring now to FIGS. 9 and 10, the tubular sheath 20 of vascular introducer 10 includes an outer sheath portion 40 and an inner sheath portion 42. The outer sheath portion 40 of the tubular sheath 20 is defined by an elongated flexible tube extruded from a soft pliable material. In an embodiment of the invention, the outer sheath portion 40 has an initial thickness T₁ of about 0.006 inches. The inner sheath portion 42 of the tubular sheath 20 is defined by a scored extrusion having a plurality of circumferentially spaced apart elongated struts 44.

The scored extrusion that defines the inner sheath portion 42 of the tubular sheath 20 is preferably formed from High-Density Polyethylene (HDPE) or a similar hard plastic material such as a Polyamide or similar Nylon material. As shown, the inner sheath portion 42 and includes twelve (12) circumferentially spaced apart elongated rigid struts 44, each separated by a longitudinally extending triangular score line or groove 46 and each one having an arcuate inner surface 48.

Those skilled in the art will readily appreciate that the number of rigid struts formed in the inner sheath portion 42 of the tubular sheath can be less than 12 or greater than 12, depending upon the dimensions and design of the sheath 20. For example, the inner sheath portion can have four (4) circumferentially spaced apart elongated rigid struts 44 to promote an even radial expansion of the outer sheath portion 40.

Preferably, the axial bore 18 of the body portion 16 and the central lumen 22 of the tubular sheath 20 is adapted and configured to transition between a first inner diameter ID₁ of 14F and a second inner diameter ID₂ of 18F. During such a transition, the outer sheath portion 40 is reduced from the initial thickness T₁ to a subsequent thickness T₂ of about 0.003 inches.

Those skilled in the art will readily appreciate the first inner diameter ID₁ of the tubular sheath 20 of vascular introducer can be greater or less than 14F, and that the second inner diameter ID₂ of the tubular sheath 20 of vascular introducer 10 can be greater or less than 18F, without departing from the spirit or scope of the subject disclosure. Moreover, it should be appreciated that sheath sizes for vascular introducers can vary from 12F or less to 24F or greater.

It is envisioned that the vascular introducer 10 of the subject invention may be provided for use and/or sale as a kit that would include an enclosure or sealed tray containing a dynamically expandable vascular introducer 10 as described above, and at least one dilator 30 having a shaft with an outer diameter OD ranging from about 14F to about 18F. Preferably, the kit would include at least one dilator 30 having a shaft 32 with an outer diameter OD of about 14F and a second dilator 30 having a shaft 32 with an OD that is greater than 14F, for example, it could have a shaft 32 with an OD of 18F. While the subject disclosure has been shown and described with reference to preferred embodiments, those skilled in the art will readily appreciate that changes or modifications may be made thereto without departing from the spirit or scope of the subject disclosure.

What is claimed is:

1. A vascular introducer comprising:
a) a proximal housing defining an axial passageway;
b) a body portion extending distally from the proximal housing and defining an axial bore in communication with the axial passageway of the proximal housing; and
c) an elongated tubular sheath extending distally from the axial bore of the body portion and defining a central lumen, wherein the axial bore of the body portion and the central lumen of the tubular sheath are adapted and configured to transition between a first inner diameter and a second inner diameter in response to introduction of a dilator having an outer diameter that is greater than the first inner diameter of the axial bore of the body portion and the central lumen of the tubular sheath.

2. A vascular introducer as recited in claim 1, wherein the proximal housing portion includes a proximal flange configured for engagement within a proximal end of the axial bore of the body portion.

3. A vascular introducer as recited in claim 1, wherein the body portion is formed from a flexible material.

4. A vascular introducer as recited in claim 1, wherein the axial bore of the body portion tapers radially inwardly in a distal direction.

5. A vascular introducer as recited in claim 1, wherein the tubular sheath includes an outer sheath portion and an inner sheath portion, the outer sheath portion defined by an elongated flexible tube and the inner sheath portion defined by a scored extrusion.

6. A vascular introducer as recited in claim 5, wherein the scored extrusion that defines the inner sheath portion of the tubular sheath is formed from a rigid plastic material.

7. A vascular introducer as recited in claim 6, wherein the scored extrusion that defines the inner sheath portion of the tubular sheath includes a plurality of circumferentially spaced apart elongated struts separated by scored longitudinal grooves.

8. A vascular introducer as recited in claim 7, wherein each of the circumferentially spaced apart elongated struts has an arcuate inner surface.

9. A vascular introducer as recited in claim 1, wherein the axial bore of the body portion and the central lumen of the tubular sheath are adapted and configured to transition between a first diameter of 14F and a second inner diameter of 18F.

10. A vascular introducer as recited in claim 1, wherein the proximal housing has an end cap that supports a hemostatic seal for sealing the axial passageway.

11. A vascular introducer system comprising:
a) a vascular introducer including:
i) a proximal housing defining an axial passageway;
ii) a body portion extending distally from the proximal housing and defining an axial bore in communication with the axial passageway of the proximal housing; and
iii) an elongated tubular sheath extending distally from the axial bore of the body portion and defining a central lumen, wherein the axial bore of the body portion and the central lumen of the tubular sheath are adapted and configured to transition between a first inner diameter and a second inner diameter in response to introduction of a dilator; and
b) at least one dilator including an obturator shaft having an outer diameter that is greater than the first inner diameter of the axial bore of the body portion and the central lumen of the tubular sheath.

12. A vascular introducer system as recited in claim 11, wherein the proximal housing portion of the vascular introducer includes a proximal flange configured for engagement within a proximal end of the axial bore of the body portion.

13. A vascular introducer system as recited in claim 11, wherein the body portion is formed from a flexible material.

14. A vascular introducer system as recited in claim 11, wherein the axial bore of the body portion tapers radially inwardly in a distal direction.

15. A vascular introducer system as recited in claim 11, wherein the tubular sheath of the vascular introducer includes an outer sheath portion and an inner sheath portion, the outer sheath portion defined by an elongated flexible tube and the inner sheath portion defined by a scored extrusion.

16. A vascular introducer system as recited in claim 15, wherein the scored extrusion that defines the inner sheath portion of the tubular sheath is formed from a rigid plastic material.

17. A vascular introducer system as recited in claim 16, wherein the scored extrusion that defines the inner sheath portion of the tubular sheath includes a plurality of circumferentially spaced apart elongated struts separated by scored longitudinal grooves.

18. A vascular introducer system as recited in claim 17, wherein each of the circumferentially spaced apart elongated rigid struts has an arcuate inner surface.

19. A vascular introducer system as recited in claim 11, wherein the axial bore of the body portion and the central lumen of the tubular sheath are adapted and configured to transition between a first diameter of 14F and a second inner diameter of 18F.

20. A vascular introducer system as recited in claim 11, wherein the proximal housing has an end cap that supports a hemostatic seal for sealing the axial passageway.
